(19) United States
(12) Patent Application Publication
(10) Pub. No.: US 2010/0267009 A1
(43) Pub. Date: Oct. 21, 2010

(54) METHOD FOR THE IN VITRO DIAGNOSIS AND/OR IN VITRO THERAPY MONITORING OF INFECTIONS

(75) Inventor: Volkmar Schollhorn, Albstadt (DE)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: AUTOIMMUN DIAGNOSTIKA GMBH, Strassberg (DE)

(21) Appl. No.: 12/740,049

(22) PCT Filed: Oct. 29, 2008
(86) PCT No.: PCT/EP08/09113

§ 371 Date: Jun. 16, 2010

(30) Foreign Application Priority Data
Oct. 29, 2007 (DE) 10 2007 052 518.6

(51) Int. Cl. C12Q 1/70; G01N 33/53

(52) U.S. Cl. 435/5; 435/7.2

(57) ABSTRACT

A method for in-vitro diagnosis and/or in-vitro therapy monitoring of infections and/or infectious diseases and differentiation between acute infections and latent or overcome infections comprising incubating eukaryotic cells with an antigen; and testing for cells (ASCs) secreting antigen-specific antibodies, the secreted antibodies of which are directed specifically against the antigen.

### RELATED APPLICATIONS

This is a §371 of International Application No. PCT/EP2008/009113, with an international filing date of Oct. 29, 2008 (WO 2009/056283 A1, published May 7, 2009), which is based on German Patent Application No. 10 2007 052 518.6, filed Oct. 29, 2007, the subject matter of which is incorporated by reference.

### TECHNICAL FIELD

This disclosure relates to a method for the in vitro diagnosis and/or in vitro therapy monitoring of infections and/or infectious diseases, in particular, to methods differentiating between acute infections on the one hand and latent or overcome infections on the other hand, and to an appropriate kit.

### BACKGROUND

For recognition of endogenous structures and for defense against exogenous organisms or substances, higher organisms, such as animals or humans, have a highly complex immune system. The human immune system can be subdivided into two groups of defense mechanisms. A differentiation is made between a humoral and a cellular immune defense. A hereditary immune defense is designated as natural immunity. If the immunity is formed only after contact with structures recognized as exogenous, ‘antigens,’ it is designated as acquired immunity. For strictly specific mechanisms of this type, leucocytes, such as monocytes, granulocytes and also T and B lymphocytes, play an important role both in cellular and in humoral defense.

In the case of infections or infectious diseases, the body is able to react to a multiplicity of antigens. As a result of a proliferation of the B lymphocytes, in particular, a clonal expansion, in combination with a selection of other defense cells including differentiation processes, the body can usually counteract the penetrated antigens with neutralizing antibodies.

In practice, for a diagnosis, a physician, however, is frequently confronted with the problem of differentiating between acute, latent or past infections or infectious diseases related thereto. A differentiation between (acute or active) infections that have just occurred and latent or chronic or even past infections is crucial, for example, for an assessment of whether infected or ill patients are still infectious.

For the differentiation of the various phases of infections or secondary diseases connected therewith, direct detection of infective agents or parts thereof in principle offers itself. However, on the one hand the difficulty exists here of isolating the infective agents. On the other hand, the isolated infective agents must be cultured with the aid of suitable culture methods such that correct detection is possible. This is usually associated with a high cost and expenditure of time and moreover cannot be carried out for all pathogens.

For an indirect form of infection detection, in principle antibodies that are formed against the infective agents are also suitable. It is disadvantageous here, however, that antibodies can usually also be detected in the blood of patients even years after an overcome infection. On account of this, in this case too, in particular, no differentiation is possible between an acute, latent or overcome infection. For possibly following therapeutic measures, however, it would be desirable if, from a diagnostic point of view, it was possible to make a statement about the infection status (acute, latent or overcome).

It could therefore be helpful to provide a reliable method for in vitro diagnosis and/or in vitro therapy monitoring, which especially allows a differentiation with respect to the infection status.

### SUMMARY

I provide a method for in vitro diagnosis and/or in vitro therapy monitoring of infections and/or infectious diseases and differentiation between acute infections and latent or overcome infections including incubating eukaryotic cells with an antigen; and testing for cells (ASCs) secreting antigen-specific antibodies, the secreted antibodies of which are directed specifically against the antigen.

I also provide a kit for in vitro diagnosis and/or in vitro therapy monitoring of infections and/or infectious diseases for differentiation between acute infections and latent or overcome infections, including at least one component for detection of antigen-specific antibody-secreting (ASCs) .

### DETAILED DESCRIPTION

The method is directed to the in vitro diagnosis and/or in vitro therapy monitoring of infections and/or infectious diseases, in particular, for differentiation between acute infections or infectious diseases on the one hand and latent or overcome infections or infectious diseases on the other hand, where eukaryotic cells are incubated, preferably on an investigation surface or on a carrier, with an antigen and tested for antigen-specific antibody-secreting cells (ASCs), the secreted antibodies of which are directed specifically against the antigen.

In other words, the method is an in vitro method for the diagnosis and/or therapy monitoring of infections and/or infectious diseases, in particular, for differentiation between acute infections or infectious diseases on the one hand and latent or overcome infections or infectious diseases on the other hand, where eukaryotic cells are incubated, preferably on an investigation surface or on a carrier, with an antigen and tested for antigen-specific antibody-secreting cells (ASCs), the secreted antibodies of which are directed specifically against the antigen.

An in vitro method is made available which, in particular, allows differentiation between acute (active) infections on the one hand and latent (chronic) or overcome infections on the other hand. In the course of the method, for this purpose eukaryotic cells are investigated for the presence of ASCs, the secreted antibodies of which are directed against the antigen that is used for the incubation of the eukaryotic cells. The ASCs that can be detected with the aid of the method are usually effector cells, in particular, effector B cells. This particular cell type allows differentiation between acute (active) infections on the one hand and latent (chronic) or overcome infections on the other hand. If the eukaryotic cells tested positive for ASCs, the eukaryotic cells originate from a human or animal body that is suffering from an acute infection of the antigen concerned or optionally also even from a past infectious disease thereof If, on the other hand, no ASCs are detected, the tested eukaryotic cells generally originate from human or animal bodies that are either suffering from a latent infection, in particular, from an infection already persisting for months, of the antigen concerned or else, from medical points of view, are regarded as healthy. A healthy human or animal body is understood as meaning a body that has either overcome an infection or else has not suffered from an infection at any point in time.

Usually, the eukaryotic cells originate from a human and/or animal sample. Preferably, the eukaryotic cells originate from samples of body fluids, in particular, blood samples, cervical smears and/or bronchial lavages.

Preferably, the eukaryotic cells, in particular, immunocompetent cells, ‘immune cells,’ occurring among the eukaryotic cells, are enriched before incubation. The eukaryotic cells are preferably blood cells, in particular, B lymphocytes, T lymphocytes, granulocytes, dendritic cells, macrophages and/or erythrocytes. For the enrichment of the eukaryotic cells, the customarily used cellular enrichment techniques are suitable. Thus, the eukaryotic cells can be enriched, for example, by centrifugation, in particular, gradient centrifugation. In the gradient centrifugation, for example, sugar gradients can be employed. Preferably, the eukaryotic cells are freed from erythrocytes before incubation. In one possible embodiment, ASCs occurring among the eukaryotic cells are enriched.

Particularly preferably, the eukaryotic cells are freed from blood serum, in particular, autologous blood serum, before incubation. This usually takes place starting from a clotted blood sample by separation of the liquid blood content from cellular blood constituents. With respect to the cellular constituents and suitable separation techniques, reference is made to the previous paragraph. By the separation of the blood serum, interfering serum constituents, in particular, serum proteins, such as, for example, albumins and/or immunoglobulins, in particular, antibodies, are removed with particular advantage. These can otherwise lead to distorted test results.

Possible antigens that can be used are allergens, mitogens and/or pathogenic types. Preferably, at least one pathogenic type, in particular, at least one infectious pathogenic type, is used as an antigen for the incubation of the eukaryotic cells. The possible antigens are generally of microbial origin, in particular, of bacterial, viral and/or fungal origin. Possible antigens of viral origin that can be used are in principle Epstein-Barr viruses, cytomegaloviruses, influenza viruses, herpes simplex viruses, mumps viruses, rubella viruses, adenoviruses, enteroviruses, Coxsackie viruses, varicella zoster viruses and/or hepatitis viruses.

In a further example, fragments of pathogenic types, in particular, pathogenic epitopes, are used as antigen. The epitopes are, in particular, peptides, preferably oligopeptides. Suitable epitopes can be composed of 5 to 25, in particular, 9 to 11, amino acid units.

It is, in particular, provided that for incubation of the eukaryotic cells at least one antigen from the group PPD, RD 1, RD 2, ESAT 6, CFP 10, MPT 41, MTB 64, PPE 44, OSP A, OSP B, OSP C, OSP D, OSP E, V1sE, p 58, p 100 Dbp A, HPV L1, HPV E1 to E7, influenza H1 to H15, HCV, HBV core AG NS2-6, Chlamydia MOMP1 and MOMP2 is used.

Particularly preferably, for the incubation of the eukaryotic cells, at least one tuberculosis-specific antigen, in particular, from the group PPD, RD 1, RD 2, MPT 64, MTB 41 and PPE 44, is used.

Further preferably, for the incubation of the eukaryotic cells, at least one borreliosis-specific antigen, in particular, from the group V1sE, OSP A, OSP B, OSP C, OSP D and OSP E, is used.

The method is usually carried out on an investigation surface suitable for this, in particular, in an investigation vessel or on a suitable carrier. The investigation surface or the carrier can be, for example, hole or cavity media of ‘hole plates,’ well plates or microtiter plates. Such hole or microtiter plates, which are preferably used as investigation vessels, are commercially obtainable, in particular, with different hole or cavity numbers (wells). For example, the process can be carried out with the aid of a 96-well microtiter plate. The microtiter plates can have, for example, cavity diameters of about 5 mm, which corresponds to a base area of approximately 20 mm². The investigation surface used is expediently level. Local concentration differences when carrying out the method can thereby be avoided. Moreover, level investigation surfaces are basically better suited for the production of cellular monolayers. Typical materials of which suitable investigation vessels can consist are, for example, polystyrene, polyvinylidene difluoride (PVDF), nitrocellulose or nylon.

In a particularly suitable example, the number of cells secreting antigen-specific antibodies is measured. With respect to the measuring methods suitable for this, reference is made to the following description.

As already mentioned, it is particularly preferred if the number of cells secreting antigen-specific antibodies is measured.

Particularly preferably, the method is carried out as the ELISPOT method (enzyme-linked immuno spot technique). In other words, it is particularly preferred if the method is an ELISPOT method. The ELISPOT method, in contrast to the ELISA method, is carried out on solid phase, usually on a suitable carrier. The carrier can be the hole plates or microtiter plates already mentioned or their hole or cavity media. With the aid of the ELISPOT method, the secretion of cytokines and/or antibodies from cells can generally be measured using antigens or antibodies that specifically bind these cytokines or antibodies. The secreted cytokines or antibodies are usually rendered visible on cavity media of microtiter plates in the form of colored dots, ‘spots.’ By enumeration of the spots and/or by determination of their color intensities by means of suitable software programs, statements can be made about the activity of the cells.

The ELISPOT method comprises, in a further example, the following steps:
a) application of capture molecules to an investigation surface or to a carrier,
b) incubation of the eukaryotic cells with the antigen on the investigation surface or on the carrier,
c) addition of detection molecules to the investigation surface or to the carrier, and
d) detection of the ASCs on the investigation surface or on the carrier, the secreted antibodies of which are directed specifically against the antigen.

The applied capture molecules can usually bind to the investigation surface and are thereby fixed to this with particular advantage. The capture molecules can, in particular, be applied to the investigation surface in layer form. The capture molecules used are preferably antibodies (capture antibodies) that are directed against antibodies or antibody subtypes of the ASCs to be detected. For example, the antibody subtypes in the case of PPD as the antigen can be at least one of the following subtypes from the group IgG1, IgG2, IgG3 and IgG4. In this connection, ‘anti-antibodies’ are also referred to. The capture molecules can be polyclonal or monoclonal antibodies, monoclonal antibodies being preferred. Preferably, for the application of the capture molecules to the investigation surface an aqueous dispersion of the capture molecules is used. For the preparation of the aqueous dispersion, the capture molecules can be dispersed in a suitable buffer, for example, an acetate buffer. Optionally, the dispersion can be filtered to obtain a solution. Usually, the aqueous dispersion is only prepared immediately before the application of the capture molecules to the investigation surface. Preferably, the capture molecules in the aqueous dispersion have a concentration between 1 and 3 μg/ml³, in particular, about 2.5 μg/ml³.

The eukaryotic cells are normally applied to the investigation surface in the form of a cell suspension. The antigen is usually applied to the investigation surface as an aqueous solution. It is, in particular, provided for the antigen to be applied to the investigation surface before the eukaryotic cells. Customarily, the antigen is fixed to the investigation surface, in particular, coupled directly to the medium of the investigation surface. In this case, no additional antigen has to be added for the incubation of the eukaryotic cells.

Preferably, the incubation of the eukaryotic cells is carried out during a period of between 2 and 24 hours, in particular, 2 and 18 hours, in particular, 2 and 4 hours. It is thereby achieved with particular advantage that memory cells are still not formed against the antigen, which would otherwise lead to a distortion of the test results. It is thus guaranteed that only the cells that are responsible for an acute or active infection are detected. By means of the incubation with the antigen, ASCs present among the eukaryotic cells, the antibodies of which are directed specifically against the antigen, are induced for the secretion of corresponding antibodies. These are captured by the capture molecules and thus bound to the investigation surface.

In addition, washing steps can be provided. The investigation surface can be washed, for example, before the addition of the detection molecules. For the washing of the investigation surface, buffer solutions, for example, phosphate buffer, are customarily used. Unbound constituents, in particular, eukaryotic cells, are thereby removed from the investigation surface. Optionally, the washing of the investigation surface can be repeated, under certain circumstances also several times.

As detection molecules, normally a further antibody type, a ‘detection antibody,’ is used. The detection molecules are likewise specifically directed against antibodies of the ASCs to be detected. Typically, the detection molecules bind, however, to other sites of the ASC antibodies than the previously described capture molecules. Ternary complexes of capture molecules, ASC antibodies and detection molecules thereby result on the investigation surface. Preferably, the detection molecules are employed as conjugate compounds. Possible suitable conjugation partners are enzymes, fluorescent dyes, gold and/or silver. Enzymes, in particular, alkaline phosphatase or horseradish peroxidase, in particular of equine origin, and/or glucose oxidase are preferred. Suitable examples of fluorescent dyes are fluorescein isothiocyanate and/or cyanine 3. In addition, however, a series of other dyes is also possible. Such dyes are adequately known to those skilled in the art. A further preferred conjugation partner is biotin. A biotinylated detection molecule is customarily used together with a conjugate compound of avidin or streptavidin and a suitable conjugation partner, for example, alkaline phosphatase or horseradish peroxidase.

Furthermore, the investigation surface can be washed before the detection of the ASCs. Unbound detection molecules can thereby be removed from the investigation surface. For the washing of the investigation surface, wash solutions, in particular buffer solutions, can be used. Optionally, the washing step can be repeated, in particular repeated several times.

Usually, the detection of the ASCs is performed by a colorimetric detection reaction. As a result of the detection reaction, colored spots (colorations) result on the investigation surface, which can optionally overlap with one another. In exceptional cases, the entire investigation surface can also be colored. The spots can be detected, in particular, counted either with a microscope or an automatic image analysis system. The comparison of the number of spots with the cell count employed allows the frequency or number of reacting cells to be calculated. Preferably, the detection reaction is catalyzed by an enzyme. The detection reaction can be carried out, for example, with the aid of suitable substrates, in particular, of chromogens. By way of example, para-nitrophenyl phosphate, carbazole or bromochloroindolyl phosphate are mentioned as suitable substrates. For example, alkaline phosphatase cleaves the phosphate radical from colorless p-nitrophenyl phosphate, whereby the yellow-colored p-nitrophenolate results. The reaction can be monitored, for example, with a photometer. The intensity of the color here is proportional to the concentration of the p-nitro-phenolate ion and, thus, also to the concentration of the antibodies of the ASCs to be detected.

Preferably, the number of cells secreting antigen-specific antibodies (ASCs) is measured. In principle, the number of spots occurring on the investigation surface can be determined. Alternatively or in combination, preferably the color intensity of individual spots or overlapping spots, optionally also of colorations that cover the entire investigation surface, is quantitatively measured. Particularly preferably, the investigation surface is broken down into a multiplicity of individual dots, the color intensity of each individual dot is measured separately and the measured intensity values are added. It can be provided for 1 to 2 million, in particular, about 1.5 million picture elements (pixels) to be determined per investigation surface. The determination of the picture elements can be carried out, for example, with the aid of a camera. For the measurement of the color intensity of an individual dot on the investigation surface, with particular advantage between 200 and 300, in particular, between 220 and 260, preferably about 256, graduations (gray scale values) are available. The processing and evaluation of the picture elements can be performed, for example, with a reader. Preferably, the total number of picture elements and, in particular, their intensity is measured with the aid of an ‘image analyzer.’ A measurement of the total coloration, based on the investigation surface or a certain part thereof, is thereby possible. An uncolored position on the investigation surface, optionally also another investigation surface, can serve as the reference value. The measure of the total activity, i.e., the total intensity of the reactions of all eukaryotic cells on the investigation surface for a defined antigen, then results from the number of excited (colored) pixels, multiplied by the color value (for example gray scale values between 0 and 256) of each excited pixel. This product is expediently divided by 1000 to obtain simply handleable numerical values (units). The determination and evaluation of the picture elements is preferably carried out from the top, i.e., above the investigation surface. Processing to give a two-dimensional image normally takes place with the aid of computer techniques.

Normally, suitable filter systems are employed for picture generation. Preferably, filter systems, in particular filter sets, with narrow-band filters are used. It can, in particular, be provided for the testing of the eukaryotic cells for ASCs to be operated with one narrow-band excitation filter and one narrow-band blocking filter per filter set used. The filter sets can be integrated together with a radiation conductor to give a filter block.

In a further example, measures can be taken whereby a coloration produced on the investigation surface is decreased in intensity per individual dot in comparison to the prior art and/or is increased in the surface area, i.e., the number of individual dots per cell. As a result of the increase in the surface area, in particular, the coloration per surface unit is weaker and, thus, coloration differences between the individual areas and/or within a larger area are more marked. In this way, the color intensities of the individual dots can be routed into the technically measurable range. Thus, also intensively colored areas, the intensity of which have exceeded the technically determinable maximum value, can be determined on account of the enlargement of the surface area. By means of the measuring method described in the preceding sections, the estimation of the infection status of a patient can additionally be improved.

The method is especially suitable for the examination of the infection status, in particular, for the differentiation of acute infections or infectious diseases on the one hand and latent or overcome infections or infectious diseases on the other hand. The designations for the characterization of the immune status can vary depending on the infections or infectious diseases. Thus, for example, in connection with tuberculosis acute and latent infections are referred to. On the other hand, in the case of the borreliosis normally the designations active and chronic borreliosis are used. Furthermore, the method is also suitable for the examination of immunization protection, i.e., with the aid of the method it can be assessed whether a still adequate immunization protection is present or a re-immunization is necessary.

The infections and/or infectious diseases are, in particular, tuberculosis, borreliosis, influenza, hepatitis A-E, herpes and/or infections and/or infectious diseases caused by cytomegaloviruses (CMV), Epstein-Barr viruses (EBV) and/or by papilloma viruses (HPV), in particular human papilloma viruses.

We further provide a kit for the in vitro diagnosis and/or in vitro therapy monitoring of infections and/or infectious diseases, in particular, for differentiation between acute infections on the one hand and latent or overcome infections on the other hand, comprising at least one component for the detection of cells (ASCs) secreting antigen-specific antibodies. Suitable kit components, in particular, are capture molecules, detection molecules, chemoluminescent dyes, fluorescent dyes and/or antigens. The components can be present in the form of aqueous dispersions and/or in dried form, for example, lyophilized form. If the kit comprises two or more components, these are preferably present spatially separate from one another. With regard to further features and properties of the kits, reference is made to the previous description.

Further details and features result from the following description of preferred forms provided as examples. The respective features can be realized on their own or multiply in combination with one another.

### EXAMPLE 1

### Detection of Tuberculosis Pathogen

A 96-well plate is used for carrying out the method. Anti-antibodies are first immobilized on the media of the cavities of the well plate as capture molecules. The anti-antibodies used here are specifically directed against the cells secreting specific antibodies against the antibodies of tuberculosis pathogen. PPD as the tuberculosis pathogen is then immobilized on the medium of the cavities. Peptides of the ‘RD1 complex’ and ‘RD2 complex’ can also be used additionally or alternatively to this. In addition, other proteins of Mycobacterium tuberculosum (38 kD, 41 kD, 44 kD, 64 kD) can also be employed. Subsequently, 100 000 to 250 000 PBMCs (peripheral blood mononuclear cells) are added per cavity. The incubation of the blood cells (together with the tuberculosis pathogens) is performed for a period of about 18 hours at about 37° C. The cells are then decanted and the well plate is washed a number of times. Subsequently, a further anti-antibody is added, which is conjugated with alkaline phosphatase (detection capture molecule). A fresh incubation is performed at about 37° C. for 4 to 12 hours. Subsequently, unbound conjugated anti-antibodies are rinsed out of the well plate medium of the 96-well plate. After addition of para-nitrophenyl phosphate, the investigation surfaces (media of the cavities) are examined for occurrence of colorations. If no colored spots (stains) occur, the reaction is negative. This means that no tuberculosis-specific plasmablasts (ASCs) are present among the blood cells. The patient whose blood cells were investigated thus has no acute or active tuberculosis. He is either negative in this regard or has latent or overcome, inactive tuberculosis.

### EXAMPLE 2

### Detection of Borrelia

A 96-well plate is used for carrying out the method. Anti-antibodies are immobilized on the media of the cavities of the well plate, which are specifically directed against the cells secreting specific antibodies against the antibodies of borrelia. Subsequently, borrelia-specific antigens, for example the peptides OSP A, OSP B, OSP C and/or V1sE of internal flagellin fragment are used. The borrelia-specific antigens are coupled to the media of the cavities with a concentration of 1 to 10 μg/ml. 100 000 to 250 000 PBMCs (peripheral blood mononuclear cells) are then added per cavity. The blood cells are incubated (together with the borrelia-specific antigens) for a period of about 18 hours at about 37° C. The blood cells are then decanted and the well plate is washed several times. Subsequently, a further anti-antibody is added, which is conjugated with alkaline phosphatase. An incubation at about 37° C. for 4 to 12 hours is again performed. Subsequently, unbound conjugated antibodies are washed from the plate. After addition of para-nitrophenyl phosphate, the media of the cavities are examined for occurrence of stains. If colored spots occur on the media of the cavities, borrelia-specific ASCs are thus detected among the blood cells examined. In this case, the diagnosis is one of acute or active borreliosis. If, on the other hand, no stains occur, this means either latent or overcome borreliosis.

### EXAMPLE 3

Detection of an Acute HPV Infection from a Cervical Smear

A 96-well plate is used for carrying out the method. Anti-antibodies are immobilized on the media of the cavities of the well plate, which are specifically directed against the cells secreting specific antibodies against the antibodies of HPV. The HPV-specific antigens used are HPV L1 and HPV E2 to E7. The HPV-specific antigens are coupled to the media of the cavities with a concentration of 1 to 10 μg/ml. 100 to 1000 cells of a washed cervical smear are then added per cavity. The cells are incubated (together with the HPV-specific antigens) for a period of about 18 hours at about 37° C. The cells are then decanted and the well plate is washed a number of times. Subsequently, a further anti-antibody is added, which is conjugated with alkaline phosphatase. An incubation at about 37° C. for 4 to 12 hours is again performed. Subsequently, unbound conjugated antibodies are washed from the plate. After addition of paranitrophenyl phosphate, the media of the cavities are examined for occurrence of stains. If colored spots occur on the media of the cavities, HPV-specific ASCs are thus detected among the cells examined. In this case, the diagnosis is one of acute or active infection with human papilloma viruses (HPV). If, on the other hand, no stains occur, this means either a latent or overcome infection.

1-13. (canceled)

14. A method for in vitro diagnosis and/or in vitro therapy monitoring of infections and/or infectious diseases and differentiation between acute infections and latent or overcome infections comprising incubating eukaryotic cells with an antigen; and testing for cells (ASCs) secreting antigen-specific antibodies, the secreted antibodies of which are directed specifically against the antigen.

15. The method as claimed in claim 14, further comprising enriching the eukaryotic cells before incubation.

16. The method as claimed in claim 14, further comprising freeing the eukaryotic cells from erythrocytes before incubation.

17. The method as claimed in claim 16, wherein the eukaryotic cells are freed from autologous blood serum before incubation.

18. The method as claimed in claim 14, wherein, for incubation of the eukaryotic cells, at least one infectious pathogenic type or an epitope thereof, is used as the antigen.

19. The method as claimed in claim 14, wherein for incubation of the eukaryotic cells, at least one antigen selected from the group consisting of PPD, RD 1, RD 2, ESAT 6, CFP 10, MPT 41, MTB 64, PPE 44, OSP A, OSP B, OSP C, OSP D, OSP E, V1sE, p 58, p 100 Dbp A, HPV L1, HPV E1 to E7, influenza H1 to H15, HCV, HBV core AG NS2-6, Chlamydia MOMP1 and MOMP2 is employed.

20. The method as claimed in claim 14, wherein, for incubation of the eukaryotic cells, at least one tuberculosis-specific antigen selected from the group consisting of PPD, RD1, RD 2, MPT 64, MTB 41 and PPE 44, is used.

21. The method as claimed in claim 14, wherein, for incubation of the eukaryotic cells, at least one borreliosis-specific antigen selected from the group consisting of V1sE, OSP A, OSP B, OSP C, OSP D and OSP E, is employed.

22. The method as claimed in claim 14, wherein the method is carried out as an ELISPOT method comprising:
a) applying capture molecules to an investigation surface,
b) incubating the eukaryotic cells with the antigen on the investigation surface,
c) adding detection molecules to the investigation surface, and
d) detecting the ASCs on the investigation surface, the secreted antibodies of which are directed specifically against the antigen.

23. The method as claimed in claim 14, wherein the infections and/or infectious diseases are selected from the group consisting of tuberculosis, borreliosis, influenza, hepatitis A-E, herpes and infections or infectious diseases caused by cytomegaloviruses (CMV), Epstein-Ban viruses (EBV) or papilloma viruses (HPV).

24. A kit for in vitro diagnosis and/or in vitro therapy monitoring of infections and/or infectious diseases for differentiation between acute infections and latent or overcome infections, comprising at least one component for detection of antigen-specific antibody-secreting (ASCs).
